(19) United States
(12) Patent Application Publication
(10) Pub. No.: US 2008/0027533 A1
(43) Pub. Date: Jan. 31, 2008

(54) Dedicated bifurcation stent apparatus and method

(75) Inventor: Randolf Von Oepen, Los Altos Hills, CA (US)

Correspondence Address: BEYER WEAVER LLP/Abbott Vascular Devices, P.O. BOX 70250, OAKLAND, CA, 94612-0250, US

(73) Assignee: Abbott Laboratories, Redwood City, CA (US)

(21) Appl. No.: 11/788,753

(22) Filed: Apr. 19, 2007

Related U.S. Application Data
(60) Provisional application No. 60/793,592, filed on Apr. 19, 2006.

(51) Int. Cl. A61F 2/06

(52) U.S. Cl. 623/1.35

(57) ABSTRACT

The present invention concerns a novel bifurcated stent apparatus for use in treating lesions at or near a bifurcation point in bifurcated vessel. More particularly, a dedicated bifurcation stent apparatus is fabricated from a single tube structure for use in a bifurcated body vessel having a main lumen and a side lumen. The stent apparatus includes a first stent portion comprised of a first stent pattern that is configured for radial expansion into a generally cylindrical main body. A second stent portion is integrally formed with the first stent portion, and includes a second stent pattern configured to form a first branch leg and a second branch leg. Collectively, the first stent portion, and the branch legs form a crocodile cut shape. Each branch leg is of a cylindrical shell-shaped arc segment in a first condition, and each of the first branch leg and the second branch leg is patterned for manipulation and radial expansion, in a second condition, into a generally cylindrical first body and a generally cylindrical second body, respectively.

### RELATED APPLICATION

This application claims priority to U.S. Provisional Patent Application No. 60/793,592 filed Apr. 19, 2006 entitled “DEDICATED BIFURCATION STENT APPARATUS AND METHOD”, which is herein incorporated by reference in its entirety.

### FIELD OF THE INVENTION

The present invention relates generally to intravascular stents designed to maintain vascular patency, and more particularly, relates to dedicated bifurcation stents.

### BACKGROUND OF THE INVENTION

A type of endoprosthesis device, commonly referred to as a stent, may be placed or implanted within a vein, artery or other tubular body organ for treating occlusions, stenoses, or aneurysms of a vessel by reinforcing the wall of the vessel or by expanding the vessel. Stents have been used to treat dissections in blood vessel walls caused by balloon angioplasty of the coronary arteries as well as peripheral arteries and to improve angioplasty results by preventing elastic recoil and remodeling of the vessel wall or prevent vulnerable plaque from rupturing. Several randomized multicenter trials have recently shown a lower restenosis rate in stent treated coronary arteries compared with balloon angioplasty alone (for example Serruys, P W et al. New England Journal of Medicine 331: 489-495, 1994, Fischman, D L et al. New England Journal of Medicine 331:496-501, 1994). Stents have been successfully implanted in the urinary tract, the bile duct, the esophagus and the tracheo-bronchial tree to scaffold those body organs, as well as implanted into the neurovascular, peripheral vascular, coronary, cardiac, and renal systems, among others. The term “stent” as used in this Application is a device that is intraluminally implanted within bodily vessels to reinforce collapsing, dissected, partially occluded, weakened, diseased or abnormally dilated or small segments of a vessel wall.

One of the drawbacks of conventional stents is that they are generally produced in a straight tubular configuration. The use of such stents to treat diseased vessels at or near a bifurcation of a vessel may create a risk of compromising the degree of patency of the primary vessel and/or its branches, and also limits the ability to insert a second stent into the side branch if the result of treatment of the primary, or main, vessel is suboptimal. Suboptimal results may occur as a result of several mechanisms, such as displacing plaque shifting, vessel spasm, dissection with or without intimal flaps, thrombosis, and embolism. In addition, the use of conventional stents to treat bifurcations requires several stents to completely cover the bifurcation vessels, which can lead to overlapping of stents or conversely, gaps between stents that prevent the achievement of adequate scaffolding.

The risk of branch compromise is increased generally in two anatomical situations. First, a side branch may be compromised when there is a stenosis in the origin of the side branch. Second, when there is an eccentric lesion in the main branch, the bifurcation site or at the carina, the expansion of a balloon or a stent can cause either plaque shifting or dissection at the side branch origin. Ballooning or stenting the side branch sequentially or with kissing balloon technology might also result in a dissection of the side branch. A common technique is to insert a balloon into the side branch through the struts of a stent deployed in the main branch spanning the bifurcation point; however, this technique carries the risk of balloon entrapment and other major complications (Nakamura, S. et al., Catheterization and Cardiovascular Diagnosis 34: 353-361 (1995)). Furthermore, it is very frequent that it is difficult to pass the stent struts deployed in the main vessel with either a balloon or a pre-mounted stent. Moreover, adequate dilation of the side branch is limited by elastic recoil of the origin of the side branch. In addition, insertion of a traditional stent into a main vessel spanning the bifurcation point may pose a limitation to blood flow and access to the side branch vessel. The term “stent jail” is often used to describe this concept. In this regard, the tubular slotted hinged design of intracoronary stents, in particular, is felt to be unfavorable for lesions with a large side branch and is generally believed to pose a higher risk of side branch vessel entrapment where the stent prevents or limits access to the side branch.

One common procedure for intraluminally implanting a stent is to first open the relevant region of the vessel with a balloon catheter and then place the stent in a position that bridges the treated portion of the vessel in order to prevent elastic recoil and restenosis of that segment. The angioplasty of the bifurcation lesion has traditionally been performed using the “kissing” balloon technique where two guidewires and two balloons are inserted, one into the main branch and the other into the side branch. Stent placement in this situation requires the removal of the guidewire from the side branch and reinsertion through the stent struts, followed by the insertion of a balloon through the struts of the stent along the guidewire.

This procedure is where the side branch wire is normally jailed by exchanging the main wire and the side branch wire. The side branch wire is taken as a guide to point the main branch wire in the right direction. This is important since the dilatation and/or stenting of the main branch might have caused plaque shift with a partial or total occlusion of the side branch. In a three dimensional setting it is hard to detect where to steer the guidewire. Nevertheless, exchanging the wires bares some risks. In situations where the shape of the main wire tip does not allow passage through the stent struts, the wire has to be removed and the tip has to be reshaped. Alternatively, a new wire has to be inserted either in addition to the already placed two wires or as an exchange for the main branch wire. It can be risky, furthermore, to remove or exchange the main vessel wire in case a dissection has occurred during the procedure. In addition, it is sometimes impossible to pass the struts of the previous implanted stent with a guide wire.

In general, when treating a bifurcation lesion using commercially available stents, it is important to cover the origin of the branch because if left uncovered, this area is prone to restenosis. In order to cover the branch origin, conventional stents inserted into the branch must protrude into the lumen of the main artery or vessel from the branch (which may cause thrombosis, again compromising blood flow). Another frequent complication experienced when stenting bifurcated vessels is the narrowing or occlusion of the origin of a side branch spanned by a stent placed in the main branch. Additionally, placement of a stent into a main vessel where the stent partially or completely extends across the opening of a branch makes future access into such branch vessels difficult if not impossible. As a result, conventional stents are often placed into the branch close to the origin, but generally not covering the origin of the bifurcation.

Accordingly, there is a need for improved stent apparatuses, most particularly for applications within the cardiac, coronary, renal, peripheral vascular, gastrointestinal, pulmonary, urinary and neurovascular systems and the brain which 1) has the ability to substantially cover the bifurcation point called carina; 2) may be used to treat lesions in one branch of a bifurcation while preserving access to the other branch for future treatment; 3) allows for differential sizing of the stents in a bifurcated stent apparatus even after the main stent is implanted; 4) may be delivered intraluminally by catheter; and 5) may be used to treat bifurcation lesions in a bifurcated vessel where the branch vessel extends from the side of the main vessel.

### SUMMARY OF THE INVENTION

The present invention is directed toward a dedicated bifurcation stent apparatus fabricated from a single tube structure for use in a bifurcated body vessel having a main lumen and a side lumen. The bifurcated stent apparatus includes a first stent portion comprised of a first stent pattern configured for radial expansion into a generally cylindrical shell-shaped main body; and a second stent portion integrally formed with the first stent portion. The second stent portion includes a second stent pattern configured to form a first branch leg and a second branch leg in a crocodile cut-shape with the first stent portion. Each branch leg is generally in the form of a cylindrical shell-shaped arc segment, in a first condition. Further, each of the first branch leg and the second branch leg is patterned for manipulation and radial expansion, in a second condition, into a generally cylindrical shell-shaped first body and a generally cylindrical shell-shaped second body, respectively.

Accordingly, a true one-piece bifurcation stent is fabricated from a single tube material without any connections, welding zones or other type of bonding. This is advantageous since any kind of connection point bares the risk of material failure.

In one specific embodiment, the first stent portion and the second stent portion are oriented in an end-to-end relationship with one another. Each branch leg of the second stent portion includes a plurality of cell segments oriented in an end-to-end manner, and each respective cell segment is integrally formed with an adjacent cell segment through one or more support links. These support links comprise transitional links and non-transitional links.

In another specific arrangement, each of the first and second branch leg includes a pair of opposed axial spines extending generally in a direction parallel to a longitudinal axis of the respective branch leg.

In yet another specific embodiment, each cell segment includes an expandable first and second strut, each having one end attached to one transitional link and the opposite end of the expandable first and second strut is attached to the other of a pair of transitional links. During manipulation of each cell segment from the first condition to the second condition, the respective second struts are inverted relative to and about the respective longitudinal axis of each branch leg, forming the substantially cylindrical-shaped branch legs.

Another specific embodiment includes each expandable first and second strut being disposed in a nested relationship, in the first condition. For instance, each nested expandable first and second strut may be sinusoidal-shaped, in the first condition.

In another aspect of the present invention, a method of fabrication of a dedicated bifurcation stent apparatus is provided, including providing a single tube structure, creating a first stent pattern in a first stent portion of the tube structure, and creating a second stent pattern in a second stent portion of the tube structure. The method further includes forming a crocodile cut shape through the second stent portion, in a generally longitudinal direction of the single tube structure. This forms a first branch leg and a second branch leg, each being generally in the shape of a cylindrical shell-shaped arc segment, in a first condition. The respective second stent portion, in the second stent pattern, of each the first and second branch legs is inverted and manipulated toward a second condition, forming a generally cylindrical shell-shape for each branch leg.

In one specific embodiment, the formation of the generally cylindrical shell-shape of each branch leg is performed through the application of a mandrel. In another configuration, the creation of the second stent pattern includes forming a plurality of cell segments oriented in an end-to-end manner for each branch leg. Each cell segment is integrally formed with a respective adjacent cell segment through a one or more support links.

In yet another arrangement, the formation of the plurality of cell segments include fabricating an expandable first and second strut, each strut having one end attached to one transitional link, and the opposite end of the expandable first and second strut attached to an opposed transitional link of a pair of transitional links. During inversion of the selected portions of each cell segment from the first condition to the second condition, the respective second strut is inverted relative to the corresponding first strut, and about the respective longitudinal axis of each branch leg.

### BRIEF DESCRIPTION OF THE DRAWINGS

The assembly of the present invention has other objects and features of advantage that will be more readily apparent from the following description of the best mode of carrying out the invention and the appended claims, when taken in conjunction with the accompanying drawing, in which:

FIG. 1 is a side elevation view, in cross-section, of a bifurcated vessel with a dedicated bifurcated stent apparatus in accordance with the present invention deployed therein.

FIG. 2 is a side elevation view of the dedicated bifurcated stent apparatus of the present invention showing the crocodile cut of the first and second branch legs, in a first condition.

FIG. 3 is another side elevation view of the dedicated bifurcated stent apparatus of the present invention showing the crocodile cut of the first and second branch legs, in the first condition.

FIG. 4 is an enlarged perspective view of an interior side of the distal end of the first branch leg of the dedicated bifurcated stent apparatus of FIG. 2, in the first condition.

FIG. 5 is an enlarged perspective view of an exterior side of the distal end of the first branch leg of the dedicated bifurcated stent apparatus of FIG. 2, in the first condition.

FIG. 6 is an end elevation view of the first branch leg of the dedicated bifurcated stent apparatus of FIG. 2, in the first condition.

FIG. 7 is a side elevation view of the dedicated bifurcated stent apparatus of the present invention showing the first and second branch legs in a second condition.

FIG. 8 is an enlarged distal end elevation view of the first branch leg of the dedicated bifurcated stent apparatus of FIG. 7, in the second condition.

FIG. 9 is a proximal end perspective view of the main body of the dedicated bifurcated stent apparatus of FIG. 7.

FIG. 10 is a side elevation view of the second stent portion of the dedicated bifurcated stent apparatus of FIG. 7 showing the first and second branch legs in the second condition.

FIG. 11 is a side elevation view of the first stent portion of dedicated bifurcated stent apparatus of FIG. 7.

FIG. 12 is an enlarged, fragmentary, interior side elevation view the first branch leg of the dedicated bifurcated stent apparatus of FIG. 4, in the first condition.

FIG. 13 is a fragmentary, interior side elevation view the first branch leg of the dedicated bifurcated stent apparatus of FIG. 12, showing inversion of the first struts of each cell segment.

FIG. 14 is a side elevation view of the first branch leg of the dedicated bifurcated stent apparatus, showing a mandrel extending through the first branch leg to form each ring segment.

FIG. 15 is a side elevation view of the first branch leg of the dedicated bifurcated stent apparatus of FIG. 14, after the mandrel has been removed.

FIG. 16 is another side elevation view of the first branch leg of the dedicated bifurcated stent apparatus of FIG. 14, showing the mandrel extending therethrough.

FIG. 17 is a side elevation view of the dedicated bifurcated stent apparatus of FIG. 14, showing the mandrel extending through a second branch leg to form each ring segment.

FIG. 18 is a side elevation view of the dedicated bifurcated stent apparatus of FIG. 14, after the mandrel has been removed from both branch legs.

FIG. 19 is another side elevation view of the dedicated bifurcated stent apparatus of FIG. 18 with the ring segments more radially expanded.

FIG. 20 is another side elevation view of the dedicated bifurcated stent apparatus of FIG. 19.

FIG. 21 is still another side elevation view of the dedicated bifurcated stent apparatus of FIG. 19.

FIG. 22 is another side elevation view of the dedicated bifurcated stent apparatus of FIG. 19 with the ring segments even more radially expanded.

FIG. 23 is another side elevation view of the dedicated bifurcated stent apparatus of FIG. 22.

FIG. 24 is a schematic side perspective view illustrating the fabrication of the dedicated bifurcated stent apparatus from a single tube beginning with an initial cut of one side into two semi tubes.

FIG. 25 is a schematic side perspective view of the single tube of FIG. 24, the two semi-tubes of which are separated to form a crocodile cut.

FIG. 26 is another schematic side perspective view of the separated semi-tubes of the single tube of FIG. 24.

FIGS. 27A-27C are a series of schematic diagrams of the upper semi-tube of FIG. 26, illustrating the cut pattern of the semi-tube and then unfolding into the stent-like scaffold.

FIG. 28 is a schematic side perspective view of a completed dedicated bifurcated stent apparatus cut from the single tube of FIG. 24.

FIG. 29 is a schematic side perspective view of a single tube illustrating the adjustable geometry of the arm lengths and diameters to meet the targeted anatomy.

### DETAILED DESCRIPTION OF THE PREFERRED EMBODIMENTS

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various figures.

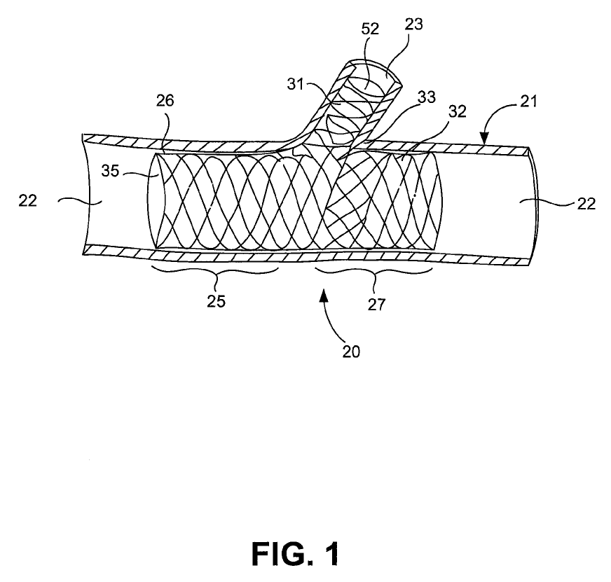

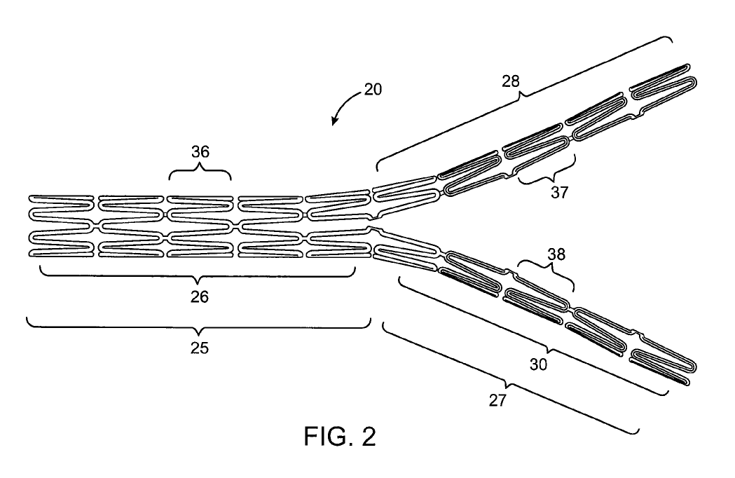

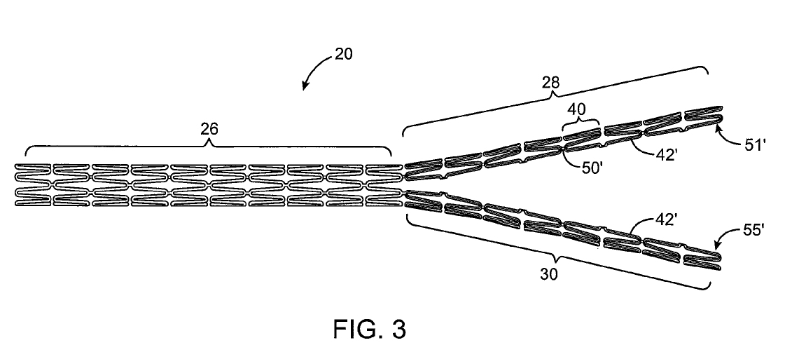

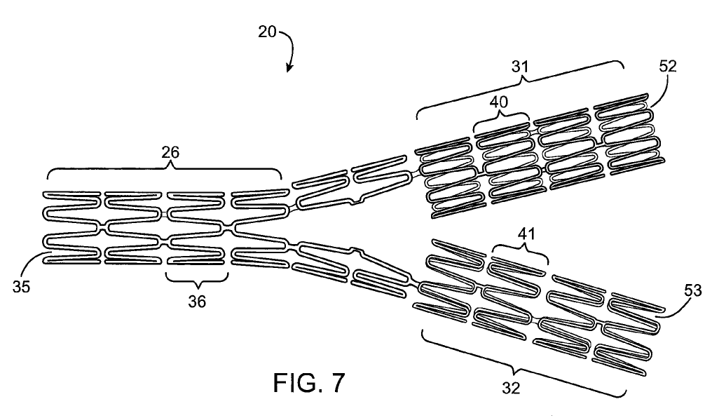

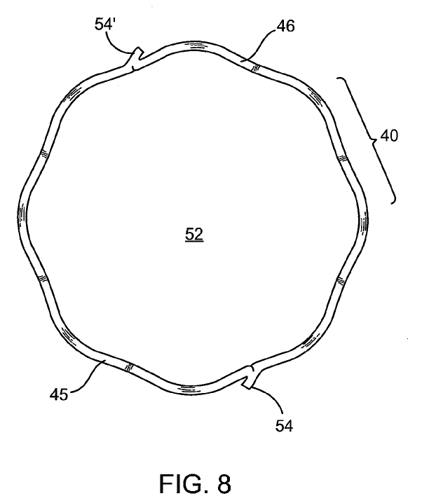

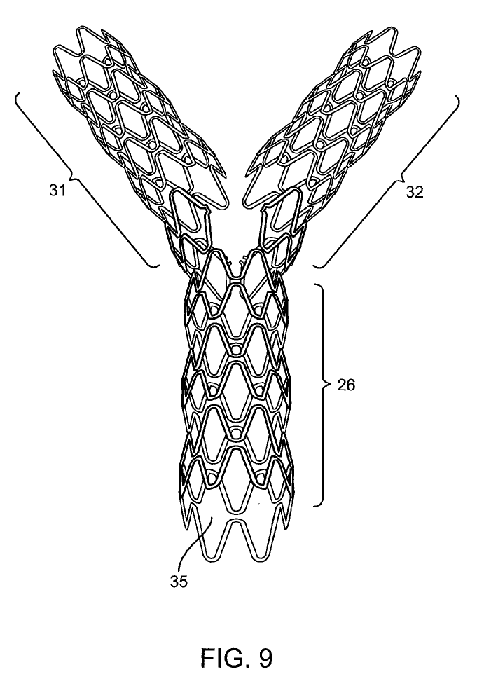

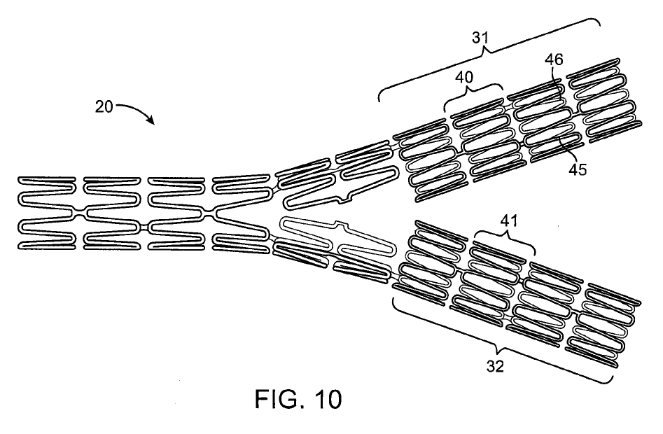

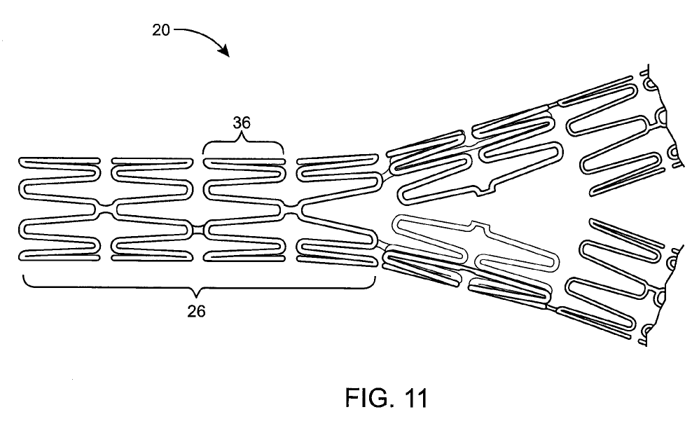

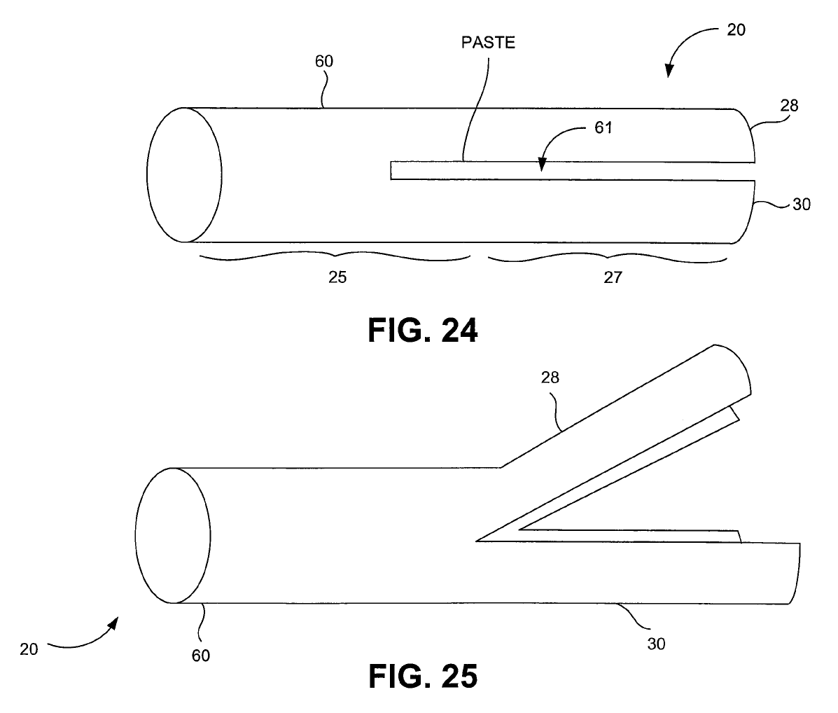

Referring now to FIGS. 1-8 and 24, a dedicated bifurcation stent apparatus, generally designated 20, is shown that is fabricated from a single tube structure 60 (FIG. 24) for use in a bifurcated body vessel 21 having a main lumen 22 and a side lumen 23. The stent apparatus 20 includes a first stent portion 25 having a first stent pattern configured for radial expansion into a generally circular cylindrical shell-shaped main body 26. The stent apparatus 20 further includes a second stent portion 27 is integrally formed with the first stent portion 25. The second stent portion 27 includes a second stent pattern configured to form a first branch leg 28 and a second branch leg 30. During either the fabrication or cut severed thereafter, the main body 26 of the first stent portion 25, and the branch legs 28, 30 forms a crocodile cut shape, wherein each branch leg is of a cylindrical shell-shaped arc segment in an initially fabricated first condition (FIGS. 2-6). Each of the first branch leg 28 and the second branch leg 30 is also patterned for manipulation and radial expansion, in a manipulated second condition, into a generally circular cylindrical shell-shaped first body 31 and a generally circular cylindrical shell-shaped second body 32, respectively (FIGS. 7-11), forming a dedicated bifurcation stent.

Accordingly, a bifurcation stent is fabricated from a single tube material without any connections, welding zones or other type of bonding. A true one-piece bifurcation stent is fabricated from a single tubular structure. This is advantageous in that any connecting points bare the risk of potential failure. Moreover, a true y-shaped bifurcation stent will avoid any struts that might be in the blood stream going into the side branch vessel, allowing continuous access to the side lumen and main lumen. Struts in the blood stream will disturb the hemodynamics and might be the potential of thrombosis or restenosis. Struts which maintain in the blood stream will not be covered by endothelial cells and therefore bare the risk that a thrombatic event might be initiated. In addition, the scaffolding that is provided by the bifurcation stent is uniform, consistent, and superior to the scaffolding results typically achieved by conventional techniques.

Using conventional techniques, such as two conventional balloon and/or modified balloons or a special dual balloon with one common proximal body and two distal sections, the bifurcated stent can be radially expanded or dilated to treat lesions at or near the carina 33 in bifurcated cardiac, coronary, renal, peripheral vascular, gastrointestinal, pulmonary, urinary and neurovascular vessels and brain vessels 22. As shown in FIGS. 1, 7 and 11, the generally cylindrical shell-shaped main body 26 of the stent apparatus 20 is configured for orientation and positioning in the main lumen 22 just proximal to the bifurcation point. Either one of the first body 31 or the second body 32 is further configured for placement distal to the bifurcation point in the main lumen 22. The other of the second body 32 or the first body 31 is positioned and deployed in the side lumen 23.

As will be apparent, the design of the present invention can be deployed in any side branch lumen angled up to 180°, but generally between about 30°-60°, from the main branch lumen. Further, the expansion geometry of the branch legs 28, 30 should be predetermined in length and diameter to accommodate most vessels. A final dilatation of the different branches can be performed to adjust it to the required diameter.

Applying conventional precision laser cutting, chemical etching, micro EDM followed by electro-polishing, if required, on a single tube material 60 (FIG. 24), the first stent portion 25 of the stent apparatus 20 can be cut and/or fabricated. Briefly, the entire fabrication process will be described in greater detail below in the discussion of FIGS. 24-29. The main body 26 of the first stent portion 25 is already generally cylindrical shell-shaped, and includes a proximal portion and a distal portion. A central passage 35 extends therethrough from the proximal end to the distal end.

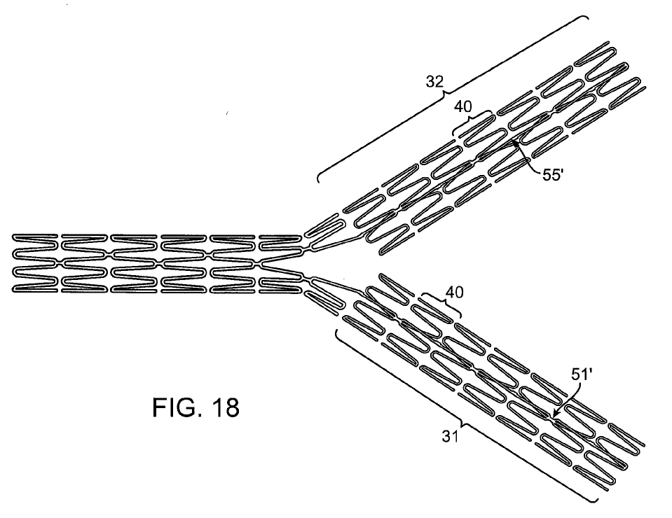

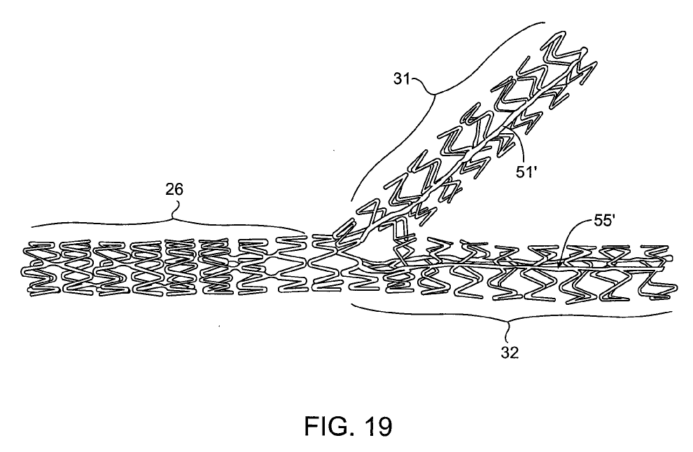

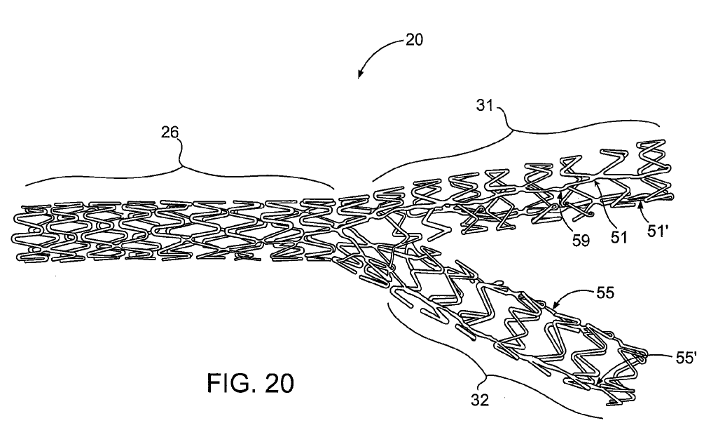

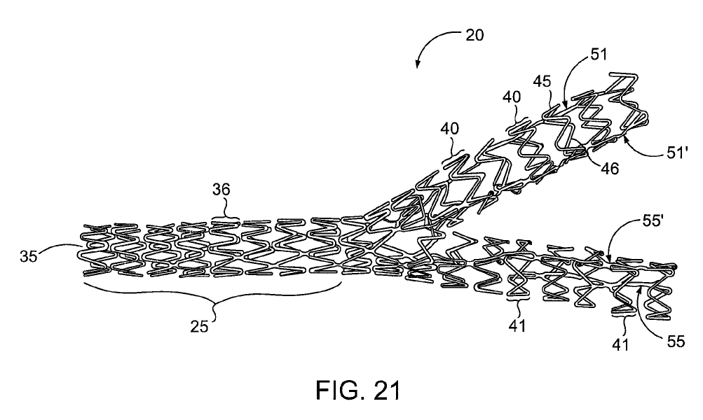

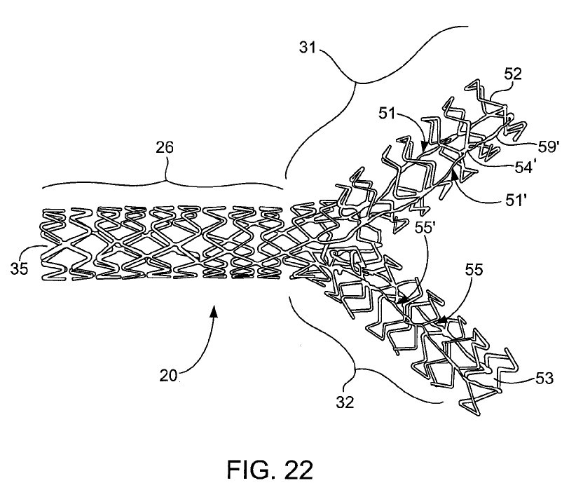

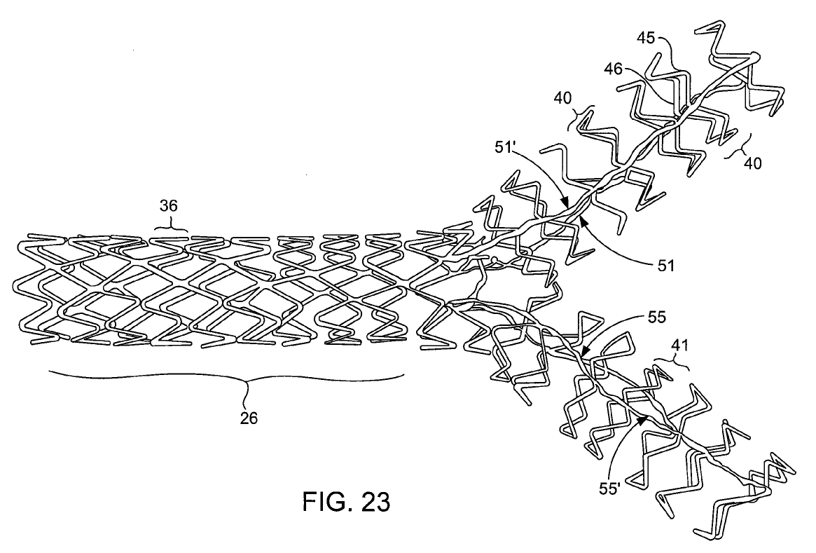

Once the basic structure of the stent has been achieved by laser cutting, etching or micro EDM or other suitable production methods, the stent can be finished by being chemically etched and/or electro-polished. The first stent pattern of the main body 26 (FIGS. 7, 11 and 18-21) is oriented at an intermediate stage that is capable of substantially greater expansion, when deployed, and of substantially greater contraction, such as when crimped around a balloon of a deployment catheter. The first stent pattern can be comprised of any conventional design capable of radial expansion and containing a plurality of joined, radially expandable main ring segments 36. For simplicity and illustration purposes, however, the first stent pattern is shown in a simple serpentine ring pattern, and is not designed to achieve optimal efficacy.

Referring now to the branch legs 28, 30 of the second stent portion 27, the respective proximal ends thereof are integrally formed with the distal end of the first stent portion 25 without any connections, weld zones or other type of bonding since the stent apparatus is fabricated from the single tube material 60 (FIG. 24). Further, in accordance with the present invention, the second stent pattern of the second stent portion 27 is designed to enable manipulation of each branch leg 28, 30, in the initial fabricated form of a cylindrical shell-shaped arc segment, in the first condition (FIGS. 2-6), into a generally cylindrical shell-shaped first and second bodies 31, 32, in the second condition (FIGS. 7-11 and 18-21). Further, each respective branch leg 28, 30 is comprised of one or more cell segments 37, 38, in the first condition, which are each manipulated into one or more branch ring segments 40, 41, in the second condition.

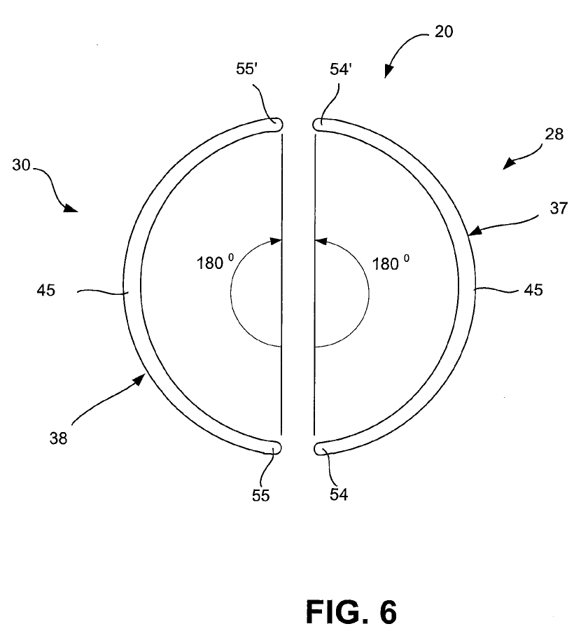

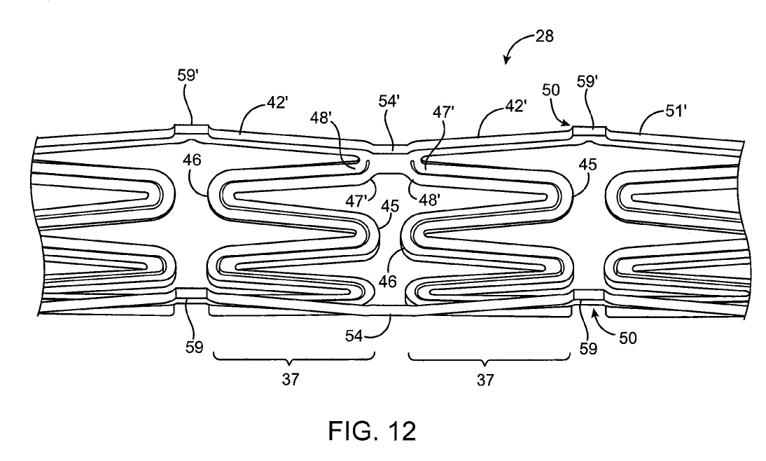

As best viewed in FIGS. 6 and 12, each branch leg 28, 30, in the first condition, is a cylindrical shell-shaped arc segment when initially fabricated from the single tube material. Although each branch leg 28, 30 is illustrated herein as two opposed semi-cylindrical shells, the arc segment of the branch leg, in the first condition, can be less than 180° as will be further described below.

Briefly, although it will be apparent that the two branch legs share a common design scaffold pattern with substantially the same characteristics, the expansion patterns of each cell segment 37, 38 (as will be described below) may differ somewhat to provide various support properties and characteristics. For the ease of description, however, only one branch leg 28 will be described in detail, and will be described in the form of a semi-cylindrical shell-shape.

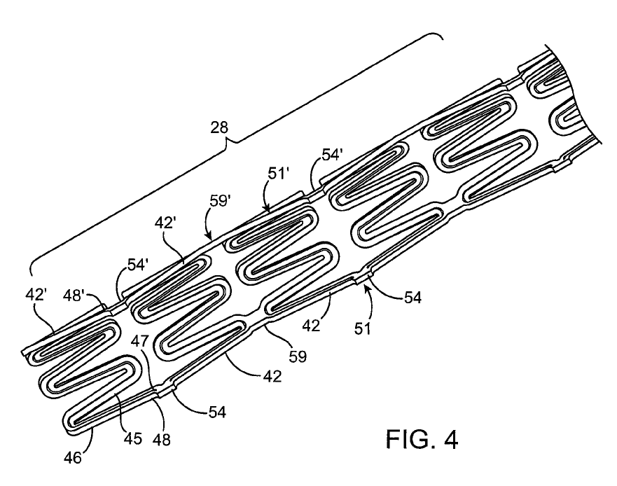

Referring now to FIGS. 4, 5 and 12, the first branch leg 28 is illustrated in the initially fabricated first condition comprising one or more cell segments 37 aligned in an adjacent side-by-side relationship. Each cell segment 37 is generally a semi-cylindrical shell inter-joined through opposed struts 42, 42′ to collectively form the first branch leg 28. The adjacent cell segments 37 are also inter-joined through one or two support links 50 interspaced between the adjacent cell segments. As will be described below, there are two types of support links, a transitional link 54, 54′ and a non-transitional link 59.

Each cell segment 37 is further composed of double cut or pair of elongated expandable struts, an elongated expandable first strut 45 and an elongated expandable second strut 46. These struts are also positioned adjacent one another in a nested manner, and are joined at their respective distal ends 47, 47′ and 48, 48′, respectively, to one another at opposed transitional links 54, 54′. In one specific embodiment, each transition link 54, 54′ supports two adjacent cell segments 37, and their respective first and second struts 45, 46.

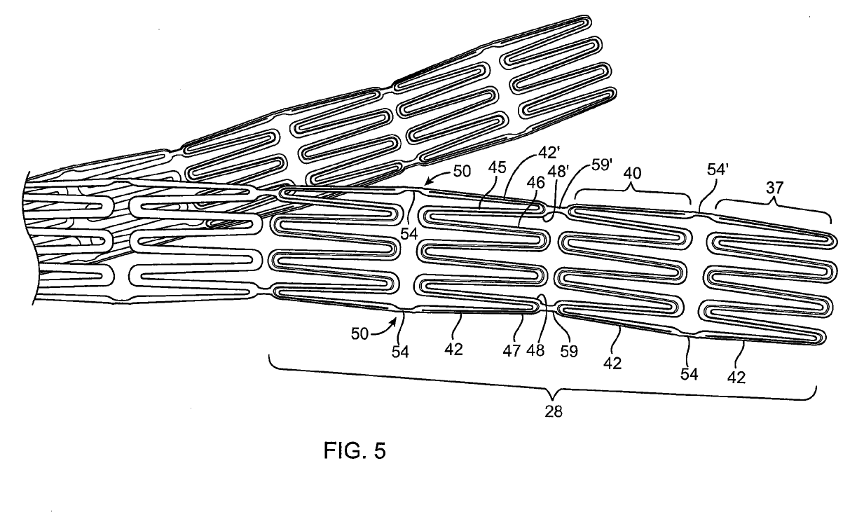

As will be described, it is the transitional links 54, 54′ that permit the inversion of one of the nested struts 45, 46 (relative the other corresponding strut), during manipulation from the first condition to the second condition, forming the substantially cylindrical branch leg. By comparison, the non-transitional links 59 are employed between selected and opposed bight portions of adjacent cell segments 37 (FIGS. 5 and 12).

In the half-cylindrical shell-shaped embodiments of FIGS. 6 and 12, the distal ends 47, 47′ and 48, 48′ of the arcuate first and second struts 45, 46, respectively, and the transitional links 54, 54′ are generally oriented about 180° apart from one another. For instance, one transitional link 54 of the cell segment is positioned at about 0°, while the opposed transition link 54′ is positioned along the arc segment at about 180° from the one transitional link 54. It will be appreciated, as mentioned, that the arc segments can be less than 180°, especially for the side branch that may be smaller than the main branch. Moreover, the arc segment can be greater than 180° as well, especially for the main branch. Furthermore, it is possible that only a short portion of crocodile cut is done over the length of the stent to assure that the side branch is created. That is, the distal part of the stent to the proximal part of the stent can nearly be formed from a cylindrical part. This last configuration would be particularly useful in the treatment of a bifurcation with disease that is localized near the carina region.

Moreover, as mentioned, each cell segment 37 (i.e., in the first condition) is integrally formed with adjacent cell segments 37 through the opposed support struts 42, 42′ (which are essentially portions of the first and second struts 45, 46 mounted to the support links 50. For example, as best viewed in FIG. 12, a proximal end of one axially extending support strut 42′ is mounted to one non-transition link 59′, respectively, of one cell segment 37, while a distal end of the support strut 42′ is mounted to one transitional link 54′, respectively, of an adjacent cell segment 37. Collectively, the alternating end-to-end joined transitional and non-transitional links 54, 59′ and the support struts 42′ cooperate to form opposed elongated axial spines 51′ that extend in a direction generally axial to the branch leg 28. The arrangement of the opposed axial spine 51 is also similarly formed. These axial spines 51, 51′ generally form the longitudinal edges of the half-cylindrical shell-shaped branch leg 28. While the lengths of each leg are shown as substantially equal, the lengths of each may be different.

In accordance with the present invention, each semi-cylindrical shell-shaped cell segment 37 (FIG. 6), in the first condition, is manipulated into a circular cylindrical shell ring segment 40 (FIG. 8), in the second condition. That is, for each cell segment 37, in the initially fabricated first condition, one of the expandable first or second struts 45, 46 of the pair is manipulated, such as by bending, to an inverted second condition. For the ease of description, the first strut 45 will be described as the one inverted strut.

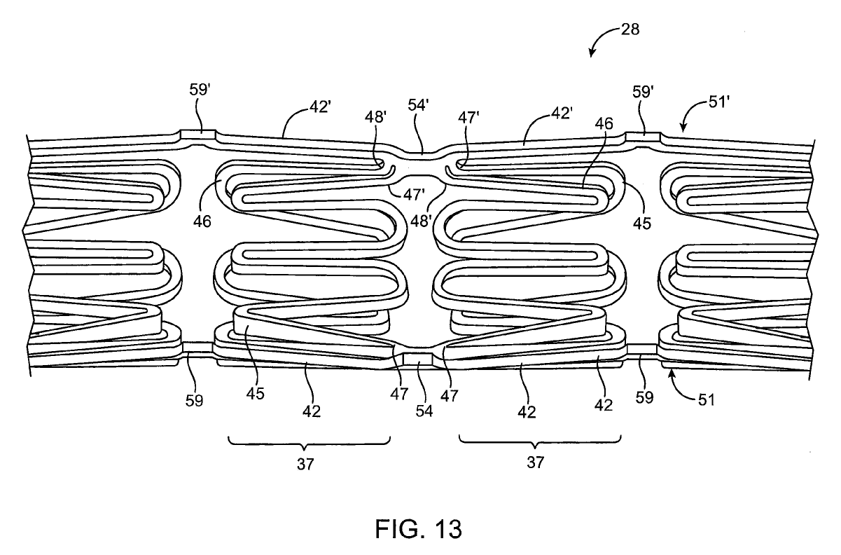

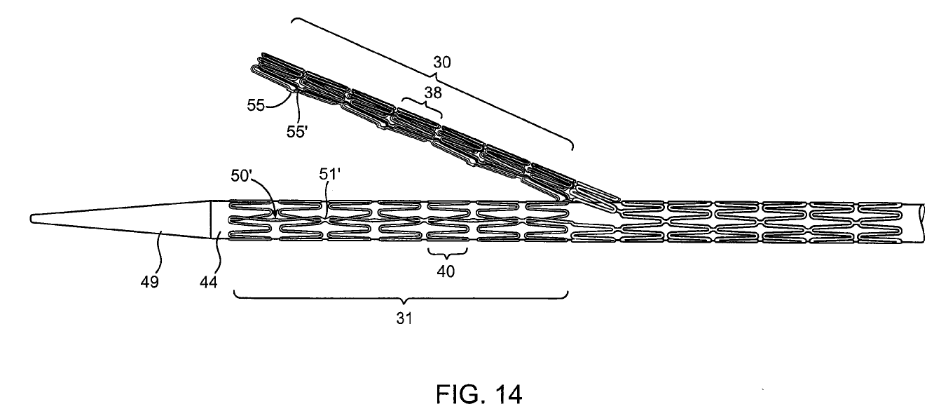

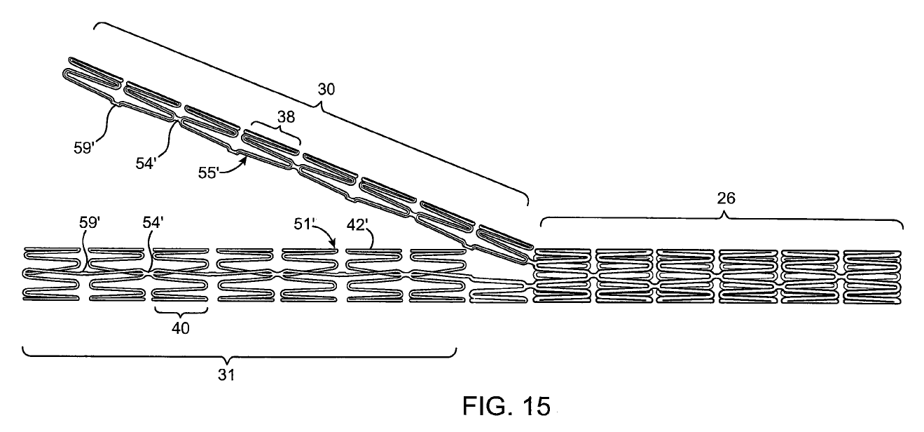

Accordingly, referring now to FIGS. 12 and 13, the first struts 45 of each cell segment 37 are carefully manipulated and separated from their corresponding second struts 46. In one example, a pair of tweezers can be applied to invert the first struts 45 by a sufficient amount from the second strut 46 (FIG. 13), so that each cell segment 37 can be formed into ring segments 40. Subsequently, an elongated rod, pin or mandrel 44 (FIGS. 14, 16 and 17) may be applied to shape the first branch leg 28 into the cylindrical shell-shaped first body 31. This is performed by feeding a tapered end 49 through the cell segments 37, and forming them into ring segments. Hence, as shown in FIG. 8, the manipulated and inverted expandable first strut 45 is generally shaped as a mirror image of the expandable second strut 46, forming a complete circle when viewed from a distal end elevation view of the first branch leg 28. The manipulation of the cells can be done before or after the stent has been polished but preferably after the stent has been annealed. For this design it might be beneficial if the stent struts are “over-polished” to achieve a mainly round cross section at the transitional links 54 and 54′. By manipulating the stent half circle into a circle, the transitional links 54, 54′ will be mechanically strained and have a round cross-section that provides a significant advantage over a square one.

Further, as shown in FIGS. 5 and 12-14, the adjacent ring segments 40, in the second position, are integrally formed together through their corresponding support links 50 (e.g., transitional links 54, 54′ or non-transitional links 59, 59′), via the support struts 42, 42′. As mentioned, these components form the common opposed axial spines 51, 51′ extending axially along the periphery of the generally circular cylindrical shell-shaped first body 31 of the first branch leg 28.

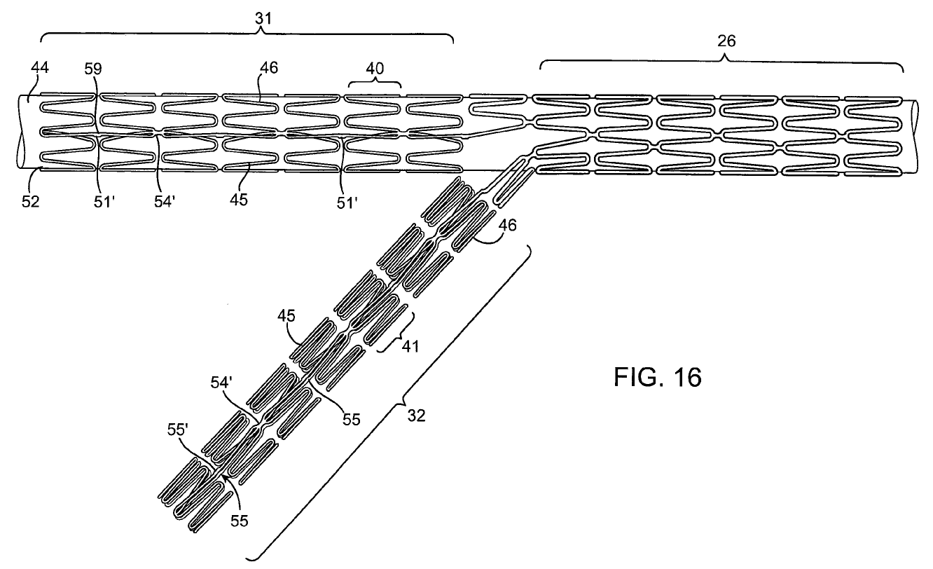

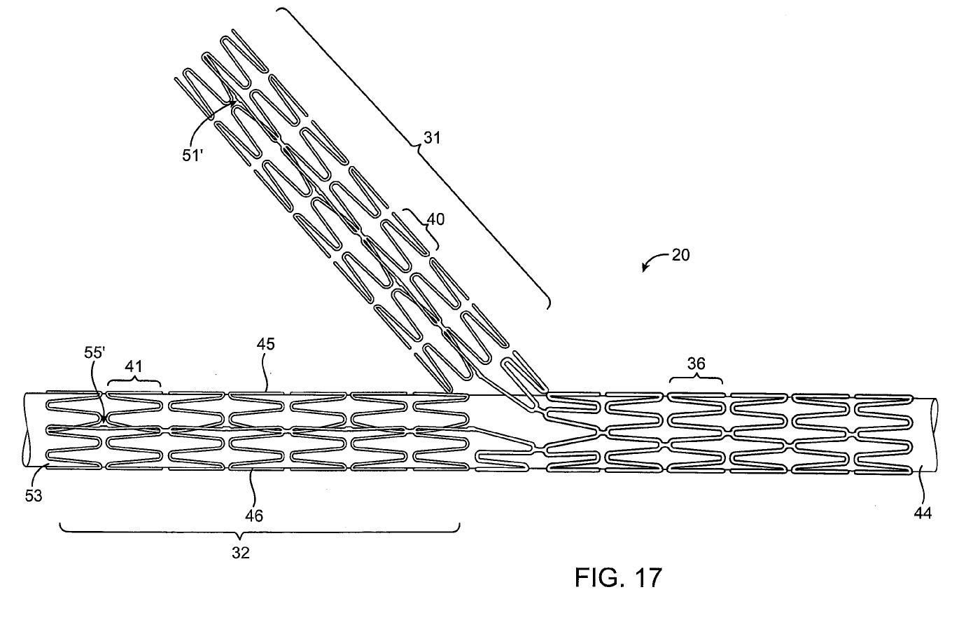

Applying a similar procedure to second branch leg 30, the expandable first strut 45 of the one or more cell segments 38 can be initially inverted and separated from the second strut 46 (FIG. 16 which illustrates the mandrel 44 still extending through the passage 52 of the first body 31). As shown in FIG. 17, the mandrel is then positioned through the passage 53 of the second branch leg 30 to form the one or more circular cylindrical shell-shaped branch ring segments 41. Consequently, the cylindrical shell-shaped second body 32 of the second branch leg 30 is created, in the second condition. Each of the first and second main bodies 31, 32 includes a proximal end and a distal end with a respective passage 52, 53, respectively extending therethrough from the respective proximal ends to the respective distal ends as to be seen in FIG. 7. Moreover, once the branch legs are manipulated into ring segments 40, 41, the branch legs 28, 30 and their respective passages 52, 53 converge at the proximal ends thereof to join and communicate with the distal end of the main body 26 and its passage 35 to create the one-piece dedicated bifurcation stent apparatus 20 of the present invention.

During the initial fabrication of the second stent portion 27, the axial spines 51, 51′ of the first branch leg 28 are oriented substantially adjacent and parallel to the corresponding axial spines 55, 55′ of the second branch leg 30, in the first condition. In one fabrication technique, the opposing axial spines 51, 55 and 51′, 55′ of each branch leg 28, 30 may be initially bridged (not shown) to one another for structural integrity during fabrication through selected support links 50. Subsequently, these bridges (i.e., support link 50) can be laser cut or etched away to separate the first branch leg 28 from the second branch leg 30, forming the crocodile cut with the first stent portion 25. In another fabrication process which will be detailed below in the discussion of FIGS. 24-29, the branch legs 28, 30 can be separated during an initial fabrication cut prior to formation of the second stent pattern

As best viewed in FIGS. 2-12, each pair of expandable struts 45, 46 of each cell segment 37, 38, in the first condition, are shown and illustrated in a simple nested sinusoidal or serpentine pattern or design. When the expandable first strut 45 of each cell segment 37, 38 is manipulated and inverted to form each ring segment, in the second condition, a simple serpentine ring pattern is formed for each of the first and second body 31, 32 for the first and second branch leg 28, 30, respectively. It will be appreciated, however, that the simple serpentine ring pattern is merely shown for illustration purposes and not to achieve optimal efficacy. For instance, more complex designs such as an Abbott Vascular stent design like a WZ, an F1, an Absolute, an Xceed, or a Vision stent pattern may be incorporated.

Different stent patterns for each cell segment 37, 38 can be fabricated. By changing the design of the stent, the stent radial force, foreshortening, expansion ratio, crimping profile, recoil and other properties of a stent can be manipulated. In most instances, the two expandable first and second struts 45, 46 for each cell segment 37, 38 will be an identical double cut nested pattern. It will be appreciated, however, that the expandable first strut 45 may differ in pattern and length than the remaining expandable second strut 46 in the cell segment 37, 38. Moreover, the width of each expandable strut 45, 46 may be the same or differ from one another. Furthermore, it is possible to adjust the wall thickness of the stent struts by i.e. grinding the tube before cutting. The strut width can be the same or differ from the width of the struts forming the main ring segments 36 of the main body 26 as well as the thickness of the different struts might vary. Depending upon the desired expansion characteristics and properties, the thicknesses and patterns of the expandable struts 45, 46 of each cell segment 37, 38 can be selected accordingly.

For instance, the inverted expandable first strut 45 may be patterned in a manner providing a true length from one distal end to the opposite distal end that is longer than that of the non-inverted expandable second strut 46 (not shown). However, when the cell segments 37 are fabricated, in the first condition, the opposed distal ends 47, 47′ of that first strut 45 are integrally formed with the common corresponding transitional links 54, 54′, and are thus separated by substantially the same true arc length or arc angle as the opposed distal ends 48, 48′ of the other non-inverted second strut 45.

In such a design, after inversion of the inverted expandable first strut 45 from the first condition, the first strut 45 can be radially expanded and deployed to cover a greater arc length than that of the other non-inverted expandable second strut 46, in the second condition. Accordingly, unlike the generally half-cylindrical shell-shaped first and second branch legs, in the first condition, where the opposed axial spines 51, 51′ are oriented apart by (an arc angle of) about 180°, this need not be the case. In such instance, in one example, one opposed axial spine 51′ (or transitional link 54′ for that matter) may be positioned in the range of about 170° to 180° from the other axial spine 51 (or transitional link 54) where inverted expandable first strut 45 will be primarily responsible for the arc length deficit during manipulation to a ring segment. Other ranges, of course, may be provided as well.

In accordance with the present invention, the material composition of the stent apparatus must be sufficiently malleable to permit inversion of the first strut 45 from the first position to the second position, enabling manipulation of the branch legs from the cell segment configuration to the ring segment configuration. In particular, either the transitional links 54, 54′, the distal ends 47, 47′ and 48, 48′ of the expandable struts 45, 46 or the support struts 42, 42′, or a combination thereof, cooperate to enable the corresponding distal ends 47, 48 and 47′, 48′ of the corresponding expandable struts 45, 46 to be inverted and substantially oppositely opposed one another at nearly 180°.

Such malleable materials should also be comprised of a non-immunoreactive material, including but not limited to any of the materials disclosed in the prior art stents that are incorporated herein by reference. One particularly suitable material, however, is (untreated) stainless steel 316L due to its excellent elongation to break. Other materials known in the art include, but are not limited to: CoCr, multiplayer material like Triflex (Stainless steel sandwiching Tantalum, metal alloys based on Tantalum, Magnesium, Niobium, Titanium Valadium etc. It is intended that the stent apparatuses of the invention may further be at least partially constructed of, or marked at certain points with, a material which may be imaged, most particularly but not limited to by x-ray and ultrasound. It is also possible to provide a beneficial coating such as an anti-restenotic, anti-thrombogenic, anti-inflammatory, or anti-proliferative agent. Of course, this is only an example, and other beneficial coating can be contemplated, for example, it could be coated with biologics, such as mesenchymal stem cells to improve vascular function.

Once the plurality of cell segments 37, 38, in the first condition, has been inverted and manipulated into a plurality of ring segments 40, 41, in the second condition, it may be desirable to relieve the stress at the inverted joints. Such inverted joints, as mentioned, could be at the transitional links 54, 54′, the distal ends 47, 47′ and 48, 48′ of the expandable struts 45, 46, the support struts 42, 42′, and/or a combination thereof.

Stress relief is preferably performed through a dedicated heat treatment process by annealing the entire stent apparatus. For example, the entire stent can be heated to its desired annealing temperture in an oven under high vacuum or protecting inert gas i.e. N2, Argon or others. After annealing, the material is more conformable and the recoil is limited to a minimum,

Once stress has been relieved through a dedicated heat treatment, the dedicated bifurcation stent apparatus 20 of the present invention may be prepared for deployment according to known methods utilizing guidewires and catheters, which are then withdrawn from the subject following deployment of the stents. The subject stents may be expanded utilizing dual balloon catheters, or by any other method currently known or developed in the future which is effective for expanding the stents of the invention. It is contemplated that prior to deployment the stents will be in a collapsed state using conventional crimping and loading techniques, and will require either mechanical expansion such as, for example, by balloon expansion upon deployment. Other methods of dilation of the stents of the present invention may exist, or may become available in the future, and such methods are contemplated as being within the scope of this invention.

From the collapsed or crimped form, the selected branch leg 28 (for example) of the stent apparatus 20 that is to be positioned in the side lumen 23 of the bifurcated vessel 21 need not be dilated to the same diameter as that disposed in the main lumen 22, of course, depending upon the diameter thereof. Further, depending upon the location of the stenosis past the bifurcation in each lumen, the operator has to choose a stent system that will fit the size about the length of the different branches as well as the diameter. The diameter can be adapted by using long stent struts or more zigzags over the diameter. The length can be adjusted by again using different strut length of varying the amount of adjacent rings. The choice of a suitable diameter and length is to be selected prior to implantation and the dedicated stent system is to be prepared according to the possible geometries.

Referring now to FIGS. 24-29, one fabrication technique for the dedicated bifurcated stent apparatus 20 will be described. As shown in FIG. 24, a single tube 60 material is provided which is longitudinally severed through the tube, forming a longitudinally extending cut 61 therethrough, and defining the lengths of the first stent portion 25 and the second stent portion 27, as well as the first branch leg 28 and the second branch leg 30 of the second stent portion. As will be mentioned, this cut 61 need extend thru the tube axis, and is dependent upon the desired diameters of the branch legs. This severing may be performed using any conventional technique, including laser cutting, etching or micro EDM.

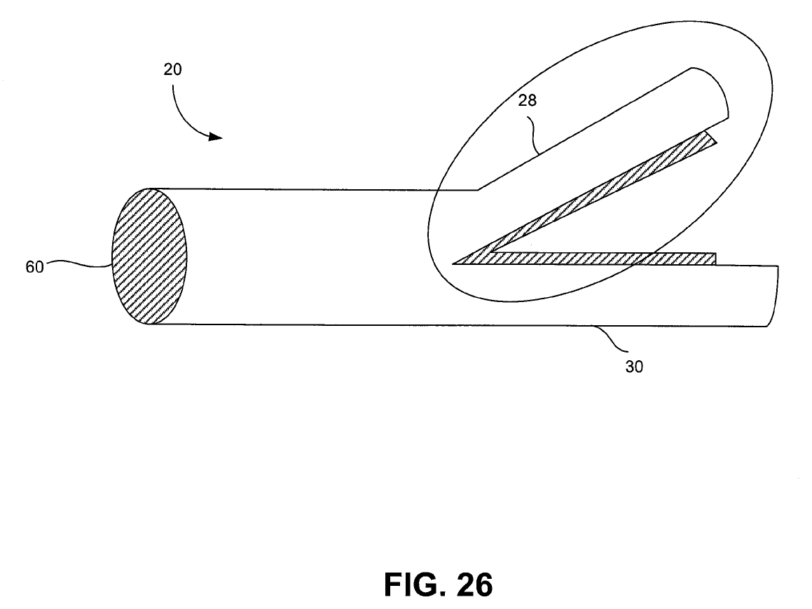

Once severed, the branch legs are separated, forming the crocodile cut shown in FIGS. 25 and 26. While only one branch leg is shown separated from the other in this illustration, both branch legs can be separated from one another as well.

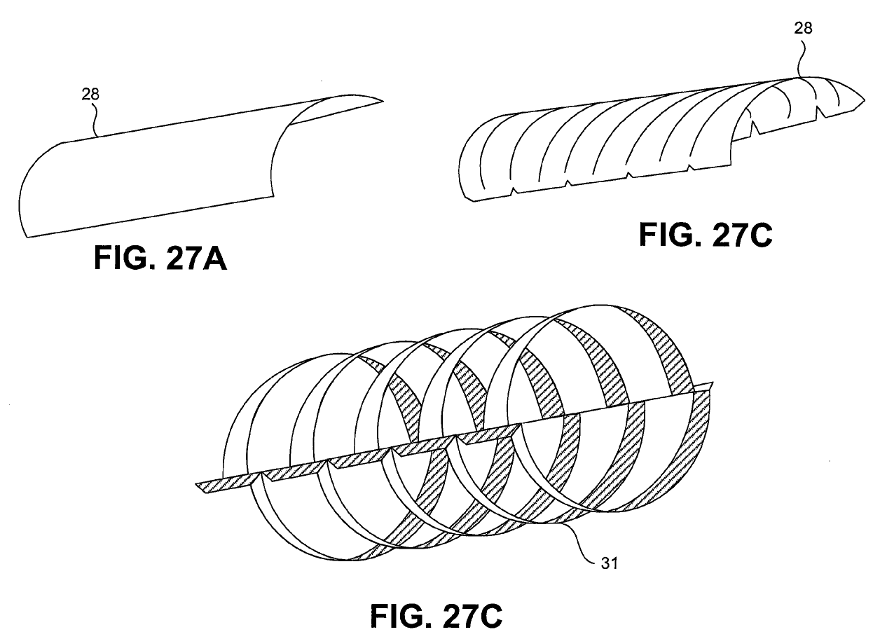

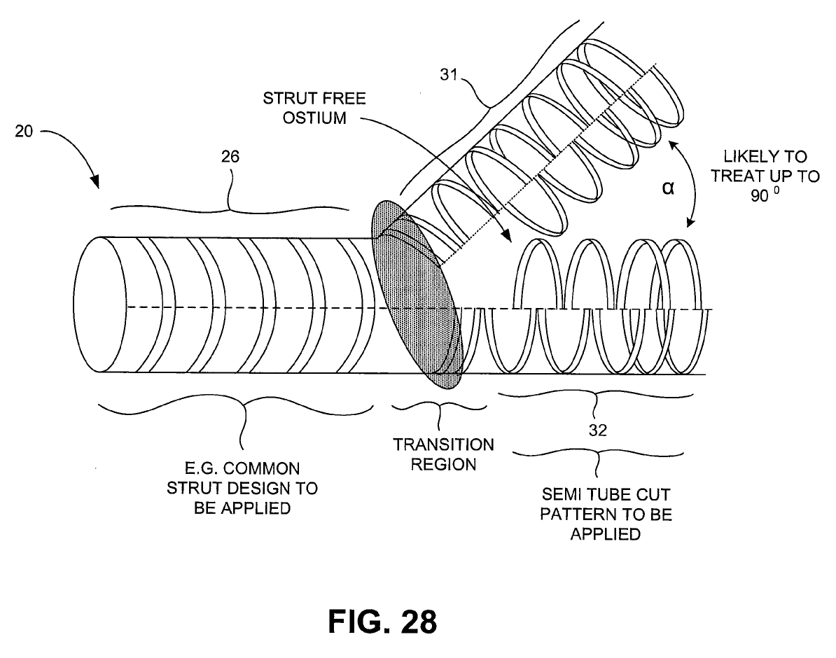

Turning now to FIGS. 27A-C, the second stent pattern is cut into the semi-cylindrical first branch leg 28 using the techniques mentioned above (FIG. 27A). It will also be appreciated that the first stent pattern can also be formed in the first stent portion 25 as well as forming the stent pattern in the second branch leg 30 (FIG. 27B). Once the second stent pattern is formed in the first branch leg, applying the mandrel 44 as shown in FIGS. 16 and 17, the semi-cylindrical branch leg can be manipulated and expanded into the stent-like scaffold of the cylindrical shell-shaped first body 31 (FIG. 27C). Applying a similar technique to the second branch leg 30, the stent-like scaffold of the cylindrical shell-shaped second body 32 can be formed, as shown in FIG. 28.

Referring now to FIG. 29, it will be appreciated that the position and length of the initial cut 61 in the single tube material 61 is very important in determining the arm lengths of the main body 26 (L1), as well as the lengths of the first body 31 (L2) and the second body 32 (L3) of the second stent portion 27.

Moreover, the position of the longitudinal cut 61 from the longitudinal axis of the single tube material 60, together with the ultimate expansion of the struts, will determine the diameters of the first body 31 (D1) and the second body 32 (D2). Depending upon the offset of the cut 61 from the tube longitudinal axis, the diameters of the first body 31 (D1) and the second body 32 (D2) can be manipulated. For example, as shown in FIG. 29, a greater offset of the cut 61 from the longitudinal axis of the tube material 60 will effectively decrease the diameter D2 of the second body 32; while simultaneously increase the diameter D1 of the first body 31. Applying these parameters, the arm lengths and diameters of the main body, the first body and the second body can be adjusted to the dimensions of the targeted anatomy.

The invention is susceptible to various modifications and alternative forms, and specific examples thereof have been shown by way of example in the drawings and are herein described in detail. For example, it is possible to construct the stent device from a shape memory material, such as Nitinol, in order to produce a self-expanding dedicated bifurcation stent. This is understood to someone skilled in the art, as well as the modifications that would be required to a delivery system in order to deploy such a stent within a patient anatomy. It should be understood, however, that the invention is not to be limited to the particular forms or methods disclosed, but to the contrary, the invention is to cover all modifications, equivalents, and alternatives falling within the spirit and scope of the claims.

What is claimed is:

1. A dedicated bifurcation stent apparatus fabricated from a single tube structure for use in a bifurcated body vessel having a main lumen and a side lumen, said stent apparatus comprising:
a first stent portion comprised of a first stent pattern configured for radial expansion into a generally cylindrical shell-shaped main body; and
a second stent portion integrally formed with said first stent portion, and having a second stent pattern configured to form a first branch leg and a second branch leg in an crocodile cut shape with said first stent portion, each branch leg being generally in the shape of a cylindrical shell-shaped arc segment, in a first condition, and each of the first branch leg and the second branch leg being patterned for manipulation and radial expansion, in a second condition, into a generally cylindrical shell-shaped first body and a generally cylindrical shell-shaped second body, respectively.

2. The dedicated bifurcation stent apparatus according to claim 1, wherein
the first stent portion and the second stent portion are oriented in an end to end relationship with one another.

3. The dedicated bifurcation stent apparatus according to claim 1, wherein
each the first and second branch leg includes a plurality of cell segments oriented in an end-to-end manner, and integrally formed with a respective adjacent cell segment through one or more respective support links.

4. The dedicated bifurcation stent apparatus according to claim 3, wherein
said support links comprise transitional links and non-transitional links.

5. The dedicated bifurcation stent apparatus according to claim 4, wherein
each the first and second branch leg includes a pair of opposed axial spines extending generally in a direction parallel to a longitudinal axis of the respective branch leg.

6. The dedicated bifurcation stent apparatus according to claim 5, wherein
each said transitional link is contained along a respective axial spine.

7. The dedicated bifurcation stent apparatus according to claim 6, wherein
each cell segment includes an expandable first and second strut, each having one end attached to one transitional link, and the opposite end of the expandable first and second strut attached to an opposed transitional link of a pair of transitional links such that during manipulation of each cell segment from the first condition to the second condition, the respective second struts are inverted relative to the first strut, and about the respective longitudinal axis of each branch leg.

8. The dedicated bifurcation stent apparatus according to claim 7, wherein
each said expandable first and second strut is disposed in a nested relationship, in the first condition.

9. The dedicated bifurcation stent apparatus according to claim 8, wherein
each nested expandable first and second strut, in the first condition, includes a plurality of U-shaped members oriented in a sinusoidal-shape.

10. The dedicated bifurcation stent apparatus according to claim 8, wherein
said non-transitional links are disposed at selected opposed bight portions of selected first and second struts of the adjacent cell segments.

11. The dedicated bifurcation stent apparatus according to claim 3, wherein
each the first and second branch leg includes a pair of opposed axial spines extending generally in a direction parallel to a longitudinal axis of the respective branch leg.

12. A dedicated bifurcation stent apparatus fabricated from a single tube structure for use in a bifurcated body vessel having a main lumen and a side lumen, said stent apparatus comprising:
a first stent portion comprised of a first stent pattern configured for radial expansion into a generally cylindrical shell-shaped main body; and
a second stent portion having a proximal end coupled to a distal end of said first stent portion, said second stent portion comprised of a second stent pattern configured to form, in a first condition, a generally semi-cylindrical shell-shaped first branch leg and a generally semi-cylindrical shell-shaped second branch leg from the single tube structure, each the first branch leg and the second branch leg patterned for manipulation and radial expansion, in a second condition, into a generally cylindrical shell-shaped first body and a generally cylindrical shell-shaped second body, respectively.

13. The dedicated bifurcation stent apparatus according to claim 12, wherein
each the first and second branch leg includes a plurality of cell segments oriented in an end-to-end manner, and integrally formed with a respective adjacent cell segment through one or more support links.

14. The dedicated bifurcation stent apparatus according to claim 13, wherein
each the first and second branch leg includes a pair of opposed axial spines extending generally in a direction parallel to a longitudinal axis of the respective branch leg.

15. The dedicated bifurcation stent apparatus according to claim 14, wherein
said support links comprise transitional links and non-transitional links, each said support link is contained along a respective axial spine.

16. The dedicated bifurcation stent apparatus according to claim 15, wherein
each cell segment includes an expandable first and second strut, each having one end attached to one transitional link, and the opposite end of the expandable first and second strut attached to an opposed transitional link of a pair of transitional links such that during manipulation of each cell segment from the first condition to the second condition, the respective second struts are inverted relative to the first strut, and about the respective longitudinal axis of each branch leg.

17. The dedicated bifurcation stent apparatus according to claim 16, wherein
each said expandable first and second strut is disposed in a nested relationship, in the first condition.

18. The dedicated bifurcation stent apparatus according to claim 17, wherein
said non-transitional links are disposed at selected opposed bight portions of selected first and second struts of the adjacent cell segments.

19. The dedicated bifurcation stent apparatus according to claim 17, wherein
each nested expandable first and second strut, in the first condition, is sinusoidal-shaped.

20. A method of fabrication of a dedicated bifurcation stent apparatus from a single tube structure comprising:
providing a single tube structure;
creating a first stent pattern in a first stent portion of the tube structure;
creating a second stent pattern in a second stent portion of the tube structure;
forming a crocodile cut shape through the second stent portion, in a generally longitudinal direction of the single tube structure, to form a first branch leg and a second branch leg, each being generally in the shape of a cylindrical shell-shaped arc segment, in a first condition;
inverting selected portions of the respective second stent portion, in the second stent pattern, of each the first and second branch legs toward a second condition; and
forming a generally cylindrical shell-shape for each branch leg.

21. The method according to claim 20, wherein,
said creating a first stent pattern, said creating a second stent pattern, and forming a crocodile cut are all performed by one of precision laser cutting, chemical etching and mirco EDM.

22. The method according to claim 20, wherein,
said forming a generally cylindrical shell-shape for each branch leg is performed through the application of a mandrel.

23. The method according to claim 20, wherein,
said creating a second stent pattern includes forming a plurality of cell segments oriented in an end-to-end manner for each branch leg, each cell segment being integrally formed with a respective adjacent cell segment through a one or more support links.

24. The method according to claim 23, wherein,
said support links comprise transitional links and non-transitional links;
and said forming a plurality of cell segments include fabricating an expandable first and second strut, each having one end attached to one transitional link, and the opposite end of the expandable first and second strut attached to an opposed transitional link of a pair of transitional links such that during the inverting the selected portions of each cell segment from the first condition to the second condition, the respective second struts are inverted relative to the first strut, and about the respective longitudinal axis of each branch leg.

25. The method according to claim 23, wherein,
said fabricating an expandable first and second strut includes disposing them in a nested relationship, in the first condition, with respect to one another.
